SEQUENCE LISTING (22 sequences)

SEQ ID NO: 1 - DNA, 22 bp, Artificial
  Synthetic DNA
ctatatcatcactgagttcatg

SEQ ID NO: 2 - DNA, 22 bp, Artificial
  Synthetic DNA
ctatatcatcattgagttcatg

SEQ ID NO: 3 - DNA, 20 bp, Artificial
  Synthetic DNA
gccagtacggggaggtgtac

SEQ ID NO: 4 - DNA, 20 bp, Artificial
  Synthetic DNA
gccagcacggggaggtgtac

SEQ ID NO: 5 - DNA, 922 bp, Homo sapiens
gcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatcygctgagcagcgggatcaatggcagcttcttggtgcgtgagagtgagagcagtcctrgccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagt

SEQ ID NO: 6 - DNA, 997 bp, Homo sapiens
gcgaacaagggcagcaaagctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaaaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccctaactacgacaagt

SEQ ID NO: 7 - DNA, 1079 bp, Homo sapiens
gtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatcygctgagcagcgggatcaatggcagcttcttggtgcgtgagagtgagagcagtcctrgccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagt

SEQ ID NO: 8 - DNA, 6104 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatcatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatgacgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcgccacgtcttcctgttcaccgagctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagagtgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatcygctgagcagcgggatcaatggcagcttcttggtgcgtgagagtgagagcagtcctrgccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctagtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctagagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgaccgttcccaggtgtatgagctgctagagaaggactaccgcatgaagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgactaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgtctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagcctggaaaggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcttgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctactcgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccgtgacagtggctgag

SEQ ID NO: 9 - DNA, 6927 bp, Homo sapiens
ggggggagggtggcggctcgatgggggagccgcctccagggggcccccccgccctgtgcccacggcgcggcccctttaagaggcccgcctggctccgtcatccgcgccgcggccacctccccccggccctccccttcctgcggcgcagagtgcgggccgggcgggagtgcggcgagagccggctggctgagcttagcgtccgaggaggcggcggcggcggcggcggcacggcggcggcggggctgtggggcggtgcggaagcgagaggcgaggagcgcgcgggccgtggccagagtctggcggcggcctggcggagcggagagcagcgcccgcgcctcgccgtgcggaggagccccgcacacaatagcggcgcgcgcagcccgcgcccttccccccggcgcgccccgccccgcgcgccgagcgccccgctccgcctcacctgccaccagggagtgggcgggcattgttcgccgccgccgccgccgcgcgggccatgggggccgcccggcgcccggggccgggctggcgaggcgccgcgccgccgctgagacgggccccgcgcgcagcccggcggcgcaggtaaggccggccgcgccatggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagaatgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaaatctgtactgcaccctggaggtggattcctttgggtattttgtgaataaagcaaagacgcgcgtctacagggacacagctgagccaaactggaacgaggaatttgagatagagctggagggctcccagaccctgaggatactgtgctatgaaaagtgttacaacaagacgaagatccccaaggaggacggcgagagcacggacagactcatggggaagggccaggtccagctggacccgcaggccctgcaggacagagactggcagcgcaccgtcatcgccatgaatgggatcgaagtaaagctctcggtcaagttcaacagcagggagttcagcttgaagaggatgccgtcccgaaaacagacaggggtcttcggagtcaagattgctgtggtcaccaagagagagaggtccaaggtgccctacatcgtgcgccagtgcgtggaggagatcgagcgccgaggcatggaggaggtgggcatctaccgcgtgtccggtgtggccacggacatccaggcactgaaggcagccttcgacgtcaataacaaggacgtgtcggtgatgatgagcgagatggacgtgaacgccatcgcaggcacgctgaagctgtacttccgtgagctgcccgagcccctcttcactgacgagttctaccccaacttcgcagagggcatcgctctttcagacccggttgcaaaggagagctgcatgctcaacctgctgctgtccctgccggaggccaacctgctcaccttccttttccttctggaccacctgaaaagggtggcagagaaggaggcagtcaataagatgtccctgcacaacctcgccacggtctttggccccacgctgctccggccctccgagaaggagagcaagctccctgccaaccccagccagcctatcaccatgactgacagctggtccttggaggtcatgtcccaggtccaggtgctgctgtacttcctgcagctggaggccatccctgccccggacagcaagagacagagcatcctgttctccaccgaagtctaaaggtcccagtccatctcctggaggcggacagatggcctggaaacctctggctaatcgggccatccgtagagcgggaaccttcctgaggtgtccttgggccacccccaagtgttgggccatctgccaagagacagcgacccaaagccgaaggacaggtggcctggaaagatcccgcccaggtctgggagccccaggctggcctcagactgtggttttttatgtggccacctgagggcgccccaagccagttcatctcggagtccaggcctggccctgggagacagggtgaagggagtggtttttatgaacttaacttagagtctaaaagatttctactggatcacttgtcaagatgcgccctctctggggagaagggaacgtgactggattccctcactgttgtatcttgaataaacgctgctgcttcatcctgtgggggccgtggccctgtccctgtgtgggtggggcctcttccatttccctgacttagaaaccacactccacttctaacagggtttgagaggcttagtcagcactgggtagcgttttgactccattcttggctttctttttctttccagaaggatttttgtgcagaaatgggtcttttgttgccatgttagtcctccttggaaggcagctcagaaggcctgtgaaatgtcaggggacaggacccccagggagggaaccccaggctacgcactttagggttcgttctccagggagagcgacctcgtcccccgatcctgaccgcccttccggcccacgctctcctgtttggcttccacaggcctggacttctctggcttctctgcccacacactccctgcccccagtgtccctgcccctgccccagcacaggtgacttcatttctgtcctctcagctcagtggactcgctcaacttttgtataagtctccacttggtggcagcagcttgctgatgacttgttttaaaactttcatcctaaataaccttttgatacttgaatatttgtaagttttatacatagtttctaatttttttccgaacagatccagatacctaataagatgctggaatgtaatccctggacaatccgtgtcctggcagcatttggtcttcctctaagcgcctggctccgctgttctcaggagtgggttctgaagtctctggagaacaggatacgtggagggttaggaaggggccaggcctagagacgggagactccctcccggagcaggtggaggcacaggaccattcgctaccccatctgccgacacctgcgggggagcccaggcattctttgtaaaccctcctgaccacctggctcaaagaaaacagaagcatggagaccgccaagtattttcaagaaataaccccatgaatattccatcacttttttagaaagaggggcttggggcaggcagaggagagaagggagagcaaactgagagccaagtttccacacggtcctgcaggaggagaggatgcagctgcccagagggaagcaggatcacatttaaggaagtgtgtggggtccctggatgacaccagcacccagtgcggctctgtctggcacccgctcccaaggtgggaggagtgggtgtcccctgtgtgtcagtgggcagctcctgctgaacccgcagctcactagggagcctgacagtggggccatgcgcctgacactcctctctgcttgtggacctggcaaggcagggagcagaaaacagccacttgaaggctttctgtctgcgtctgtgtgcagtgtggatttagttgtgcttttttcttgctgggagagcacagccaccatttacaagcagtgtcaccgtcgtgggtggcgaggacagaacaggagcctctgctctctgtacctatctgggcccggtgggctcccttgtcctggcttccatctctgtctcagcgaccattcagccctgcgcaggaacacgtgttgcttagaaaagccaaatccagccttgtctctgcctcctctggtctcatgatgtgcatctgttaccttgaaactggaaaccagtctatcaatgtctgtgccaattttttattccctccccaacctccttccccatacgactttttatttatgtaggatgtgtgctgtctaatgatgggatgaccacacttttccatgttctaaaagtgctcctctcccgcagggtcccagggctggtggttgctttgggtctacagctacgtcttacccacctcctgcctcaacagcctgtgtggtggcaaagccggtgtggggctggggaacgcagcgttctccaggagggggacctggctctccttctgcaatgcaggcgaaggcctagatgccagtgtgacctcccacaaggcgtggcttccagactccccggccggaagtgatgcttttttgccttgggccctgggtttgaagcagcctggctttctcttggtaagtggctggtgtcttagcagctgcaatctgagctcagccacctacacaccaccgtggccgacactttcattaaaaagtttcctgagacga

SEQ ID NO: 10 - DNA, 6795 bp, Homo sapiens
ggggggagggtggcggctcgatgggggagccgcctccagggggcccccccgccctgtgcccacggcgcggcccctttaagaggcccgcctggctccgtcatccgcgccgcggccacctccccccggccctccccttcctgcggcgcagagtgcgggccgggcgggagtgcggcgagagccggctggctgagcttagcgtccgaggaggcggcggcggcggcggcggcacggcggcggcggggctgtggggcggtgcggaagcgagaggcgaggagcgcgcgggccgtggccagagtctggcggcggcctggcggagcggagagcagcgcccgcgcctcgccgtgcggaggagccccgcacacaatagcggcgcgcgcagcccgcgcccttccccccggcgcgccccgccccgcgcgccgagcgccccgctccgcctcacctgccaccagggagtgggcgggcattgttcgccgccgccgccgccgcgcgggccatgggggccgcccggcgcccggggccgggctggcgaggcgccgcgccgccgctgagacgggccccgcgcgcagcccggcggcgcaggtaaggccggccgcgccatggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagaatgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaaatctgtactgcaccctggaggtggattcctttgggtattttgtgaataaagcaaagacgcgcgtctacagggacacagctgagccaaactggaacgagctggacccgcaggccctgcaggacagagactggcagcgcaccgtcatcgccatgaatgggatcgaagtaaagctctcggtcaagttcaacagcagggagttcagcttgaagaggatgccgtcccgaaaacagacaggggtcttcggagtcaagattgctgtggtcaccaagagagagaggtccaaggtgccctacatcgtgcgccagtgcgtggaggagatcgagcgccgaggcatggaggaggtgggcatctaccgcgtgtccggtgtggccacggacatccaggcactgaaggcagccttcgacgtcaataacaaggacgtgtcggtgatgatgagcgagatggacgtgaacgccatcgcaggcacgctgaagctgtacttccgtgagctgcccgagcccctcttcactgacgagttctaccccaacttcgcagagggcatcgctctttcagacccggttgcaaaggagagctgcatgctcaacctgctgctgtccctgccggaggccaacctgctcaccttccttttccttctggaccacctgaaaagggtggcagagaaggaggcagtcaataagatgtccctgcacaacctcgccacggtctttggccccacgctgctccggccctccgagaaggagagcaagctccctgccaaccccagccagcctatcaccatgactgacagctggtccttggaggtcatgtcccaggtccaggtgctgctgtacttcctgcagctggaggccatccctgccccggacagcaagagacagagcatcctgttctccaccgaagtctaaaggtcccagtccatctcctggaggcggacagatggcctggaaacctctggctaatcgggccatccgtagagcgggaaccttcctgaggtgtccttgggccacccccaagtgttgggccatctgccaagagacagcgacccaaagccgaaggacaggtggcctggaaagatcccgcccaggtctgggagccccaggctggcctcagactgtggttttttatgtggccacctgagggcgccccaagccagttcatctcggagtccaggcctggccctgggagacagggtgaagggagtggtttttatgaacttaacttagagtctaaaagatttctactggatcacttgtcaagatgcgccctctctggggagaagggaacgtgactggattccctcactgttgtatcttgaataaacgctgctgcttcatcctgtgggggccgtggccctgtccctgtgtgggtggggcctcttccatttccctgacttagaaaccacactccacttctaacagggtttgagaggcttagtcagcactgggtagcgttttgactccattcttggctttctttttctttccagaaggatttttgtgcagaaatgggtcttttgttgccatgttagtcctccttggaaggcagctcagaaggcctgtgaaatgtcaggggacaggacccccagggagggaaccccaggctacgcactttagggttcgttctccagggagagcgacctcgtcccccgatcctgaccgcccttccggcccacgctctcctgtttggcttccacaggcctggacttctctggcttctctgcccacacactccctgcccccagtgtccctgcccctgccccagcacaggtgacttcatttctgtcctctcagctcagtggactcgctcaacttttgtataagtctccacttggtggcagcagcttgctgatgacttgttttaaaactttcatcctaaataaccttttgatacttgaatatttgtaagttttatacatagtttctaatttttttccgaacagatccagatacctaataagatgctggaatgtaatccctggacaatccgtgtcctggcagcatttggtcttcctctaagcgcctggctccgctgttctcaggagtgggttctgaagtctctggagaacaggatacgtggagggttaggaaggggccaggcctagagacgggagactccctcccggagcaggtggaggcacaggaccattcgctaccccatctgccgacacctgcgggggagcccaggcattctttgtaaaccctcctgaccacctggctcaaagaaaacagaagcatggagaccgccaagtattttcaagaaataaccccatgaatattccatcacttttttagaaagaggggcttggggcaggcagaggagagaagggagagcaaactgagagccaagtttccacacggtcctgcaggaggagaggatgcagctgcccagagggaagcaggatcacatttaaggaagtgtgtggggtccctggatgacaccagcacccagtgcggctctgtctggcacccgctcccaaggtgggaggagtgggtgtcccctgtgtgtcagtgggcagctcctgctgaacccgcagctcactagggagcctgacagtggggccatgcgcctgacactcctctctgcttgtggacctggcaaggcagggagcagaaaacagccacttgaaggctttctgtctgcgtctgtgtgcagtgtggatttagttgtgcttttttcttgctgggagagcacagccaccatttacaagcagtgtcaccgtcgtgggtggcgaggacagaacaggagcctctgctctctgtacctatctgggcccggtgggctcccttgtcctggcttccatctctgtctcagcgaccattcagccctgcgcaggaacacgtgttgcttagaaaagccaaatccagccttgtctctgcctcctctggtctcatgatgtgcatctgttaccttgaaactggaaaccagtctatcaatgtctgtgccaattttttattccctccccaacctccttccccatacgactttttatttatgtaggatgtgtgctgtctaatgatgggatgaccacacttttccatgttctaaaagtgctcctctcccgcagggtcccagggctggtggttgctttgggtctacagctacgtcttacccacctcctgcctcaacagcctgtgtggtggcaaagccggtgtggggctggggaacgcagcgttctccaggagggggacctggctctccttctgcaatgcaggcgaaggcctagatgccagtgtgacctcccacaaggcgtggcttccagactccccggccggaagtgatgcttttttgccttgggccctgggtttgaagcagcctggctttctcttggtaagtggctggtgtcttagcagctgcaatctgagctcagccacctacacaccaccgtggccgacactttcattaaaaagtttcctgagacga

SEQ ID NO: 11 - DNA, 3840 bp, Homo sapiens
ggccttccccctgcgaggatcgccgttggcccgggttggctttggaaagcggcggtggctttgggccgggctcggcctcgggaacgccaggggcccctgggtgcggacgggcgcggccaggagggggttaaggcgcaggcggcggcggggcgggggcgggcctggcgggcgccctctccgggccctttgttaacaggcgcgtcccggccagcggagacgcggccgccctgggcgggcgcgggcggcgggcggcggtgagggcggcctgcggggcggcgcccgggggccgggccgagccgggcctgagccgggcccggaccgagctgggagaggggctccggcccgatcgttcgcttggcgcaaaatgttggagatctgcctgaagctggtgggctgcaaatccaagaaggggctgtcctcgtcctccagctgttatctggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatcygctgagcagcgggatcaatggcagcttcttggtgcgtgagagtgagagcagtcctrgccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctagtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctagagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgaccgttcccaggtgtatgagctgctagagaaggactaccgcatgaagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgactaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgtctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagcctggaaaggcagtgccttagggacccctgctgcagctgagccagtgacccccaccagcaaagcaggctcaggtgcaccaaggggcaccagcaagggccccgccgaggagtccagagtgaggaggcacaagcactcctctgagtcgccagggagggacaaggggaaattgtccaagctcaaacctgccccgccgcccccaccagcagcctctgcagggaaggctggaggaaagccctcgcagaggcccggccaggaggctgccggggaggcagtcttgggcgcaaagacaaaagccacgagtctggttgatgctgtgaacagtgacgctgccaagcccagccagccggcagagggcctcaaaaagcccgtgctcccggccactccaaagccacaccccgccaagccgtcggggacccccatcagcccagcccccgttcccctttccacgttgccatcagcatcctcggccttggcaggggaccagccgtcttccactgccttcatccctctcatatcaacccgagtgtctcttcggaaaacccgccagcctccagagcgggccagcggcgccatcaccaagggcgtggtcttggacagcaccgaggcgctgtgcctcgccatctctgggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgtcagcaggcagtggtccggcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccatgacagtggctgag

SEQ ID NO: 12 - DNA, 30 bp, Artificial
  Synthetic DNA
ccggaattctccgctgaccatcaataagga

SEQ ID NO: 13 - DNA, 38 bp, Artificial
  Synthetic DNA
ataagaatgcggccgcacgtcggacttgatggagaact

SEQ ID NO: 14 - DNA, 36 bp, Artificial
  Synthetic DNA
ccccgttctatatcatcattgagttcatgacctacg

SEQ ID NO: 15 - DNA, 36 bp, Artificial
  Synthetic DNA
cgtaggtcatgaactcaatgatgatatagaacgggg

SEQ ID NO: 16 - DNA, 38 bp, Artificial
  Synthetic DNA
caagctgggcgggggccagcacggggaggtgtacgagg

SEQ ID NO: 17 - DNA, 38 bp, Artificial
  Synthetic DNA
cctcgtacacctccccgtgctggcccccgcccagcttg

SEQ ID NO: 18 - DNA, 25 bp, Artificial
  Synthetic DNA
cccttcagcggccagtagcatctga

SEQ ID NO: 19 - DNA, 21 bp, Artificial
  Synthetic DNA
tccgctgaccatcaayaagga

SEQ ID NO: 20 - DNA, 22 bp, Artificial
  Synthetic DNA
acgtcggacttgatggagaact

SEQ ID NO: 21 - DNA, 23 bp, Artificial
  Synthetic DNA
aagaccttgaaggaggacaccat

SEQ ID NO: 22 - DNA, 22 bp, Artificial
  Synthetic DNA
acgtcggacttgatggagaact

(19) United States
(12) Patent Application Publication
(10) Pub. No.: US 2017/0253932 A1
(43) Pub. Date: Sep. 7, 2017

(54) METHOD FOR DETECTING BCR-ABL INHIBITOR RESISTANCE-RELATED MUTATION AND DATA ACQUISITION METHOD FOR PREDICTING BCR-ABL INHIBITOR RESISTANCE USING THE SAME

(71) Applicants: Toyo Kohan Co., Ltd., Tokyo (JP); Yamaguchi University, Yamaguchi (JP)

(72) Inventors: Mitsuyoshi OBA, Yamaguchi (JP); Kenichi ABE, Yamaguchi (JP); Hirofumi YAMANO, Yamaguchi (JP); Toshiaki YUJIRI, Yamaguchi (JP)

(73) Assignees: Toyo Kohan Co., Ltd., Tokyo (JP); Yamaguchi University, Yamaguchi (JP)

(21) Appl. No.: 15/519,671

(22) PCT Filed: Oct. 16, 2015
(86) PCT No.: PCT/JP2015/079267

§ 371 Date: Apr. 17, 2017

(30) Foreign Application Priority Data
Oct. 17, 2014 (JP) 2014-212228

(51) Int. Cl. C12Q 1/68; G06F 19/18

(52) U.S. Cl. CPC C12Q 1/6886; C12Q 1/6853; G06F 19/18; C12Q 2600/156; C12Q 2600/158

(57) ABSTRACT

An object of the present invention is to detect a mutation related to BCR-ABL inhibitor resistance with high accuracy for chronic myelogenous leukemia or the like. A method for detecting the mutation comprises the steps of: amplifying a region including a fusion site and a mutation site related to BCR-ABL inhibitor resistance contained in a BCR-ABL fusion gene using a biological sample collected from a subject; and genotyping of the mutation site related to BCR-ABL inhibitor resistance using nucleic acids amplified in the above step.

### TECHNICAL FIELD

The present invention relates to a method for detecting a BCR-ABL inhibitor resistance-related mutation in chronic myelogenous leukemia or the like and a data acquisition method for predicting BCR-ABL inhibitor resistance using the same.

### BACKGROUND ART

In most of chronic myelogenous leukemia (CML) cases, a gene mutation called the Philadelphia chromosome is observed. The Philadelphia chromosome is derived from a translocation between the long arm of chromosome 9 and the long arm of chromosome 22. As a result of this translocation, the BCR (breakpoint cluster region) gene region on chromosome 22 and the ABL, gene region on chromosome 9 are fused, resulting in a BCR-ABL fusion (chimera) gene. A BCR-ABL fusion protein encoded by this fusion gene constantly promotes tyrosine kinase activity, and therefore, it is regarded as a key factor of chronic myelogenous leukemia.

Meanwhile, BCR-ABL inhibitors represented by imatinib are currently used as molecular targeting therapeutic agents for the BCR-ABL fusion protein in the cases of chronic myelogenous leukemia (CML), Philadelphia chromosome-positive acute lymphoblastic leukemia (Ph⁺ ALL), and KIT (CD117)-positive gastrointestinal stromal tumor (GIST). However, it is known that many patients in the advanced stage of chronic myelogenous leukemia have good response to BCR-ABL inhibitor treatment, which causes a decrease in the expression level of the BCR-ABL fusion gene, while on the other hand, once a mutation occurs in the BCR-ABL fusion gene during treatment, treatment resistance is manifested. Examples of this mutation as a mutation showing imatinib treatment resistance include a mutation at position 253 from tyrosine (wild type) to phenylalanine or histidine (mutant type), a mutation at position 255 from glutamic acid (wild type) to lysine or valine (mutant type), a mutation at position 315 from tyrosine (wild type) to isoleucine (mutant type) (Non-Patent Literature 1).

Non-Patent Literature 1 discloses that among the above mutations, the mutations in the BCR-ABL kinase domain other than the mutation at position 315 from tyrosine (wild type) to isoleucine (mutant type) show sensitivity to nilotinib or dasatinib, which is a BCR-ABL inhibitor. Therefore, if the presence or absence of a mutation in the BCR-ABL kinase domain related to BCR-ABL inhibitor resistance can be evaluated at once, it becomes possible to take measures to make a treatment strategy or change a treatment method. Hence, there has been a demand to detect a mutation related to BCR-ABL inhibitor resistance described above in a convenient manner with good accuracy.

### CITATION LIST

### Non-Patent Literature

Non-Patent Literature 1: O'Hare T, Eide C A, Deininger M W. Bcr-Abl kinase domain mutations, drug resistance, and the road to a cure for chronic myeloid leukemia. Blood 2007; 110: 2242-2249.

### SUMMARY OF INVENTION

### Technical Problem

However, a biological sample collected from a subject sometimes contains a mixture of cells in which the BCR-ABL fusion gene is expressed and normal cells in which the ABL gene is expressed without the above translocation. Therefore, in the case of detection of a mutation related to BCR-ABL inhibitor resistance, a mutation in the ABL kinase domain on chromosome 9 from normal cells contained in the biological sample can be detected. Since the mutation in the ABL kinase domain does not directly attribute to BCR-ABL inhibitor resistance, the mutation related to BCR-ABL inhibitor resistance may not be correctly detected, which has been problematic. Therefore, an object of the present invention is to provide a method for detecting a BCR-ABL inhibitor resistance-related mutation, whereby a mutation related to BCR-ABL inhibitor resistance can be detected with high accuracy for chronic myelogenous leukemia or the like and a data acquisition method for predicting BCR-ABL inhibitor resistance using the same.

### Solution to Problem

As a result of intensive studies in order to achieve the above object, the present inventors found that it is possible to specifically amplify a BCR-ABL fusion gene generated through translocation and detect a mutation related to BCR-ABL inhibitor resistance contained in amplified nucleic acids, thereby specifically detecting the mutation. This has led to the completion of the present invention.

The present invention encompasses the following.

(1) A method for detecting a BCR-ABL inhibitor resistance-related mutation, comprising the steps of:

amplifying a region including a fusion site and a mutation site related to BCR-ABL inhibitor resistance contained in a BCR-ABL fusion gene using a biological sample collected from a subject; and

genotyping of the mutation site related to BCR-ABL inhibitor resistance using nucleic acids amplified in the above step.

(2) The method for detecting a BCR-ABL inhibitor resistance-related mutation according to (1), wherein abundance ratio of wild-type and/or mutation-type among the amplified nucleic acids is calculated using a wild-type probe for wild-type and a mutant-type probe for mutant-type for the mutation site in the step of genotyping.
(3) The method for detecting a BCR-ABL inhibitor resistance-related mutation according to (2), wherein the abundance ratio of wild-type is defined as a proportion of the amount of nucleic acids that specifically hybridize to the wild-type probe and the abundance ratio of mutant-type is defined as a proportion of the amount of nucleic acids that specifically hybridize to the mutant-type probe with respect to the sum of the amount of nucleic acids that specifically hybridize to the wild-type probe and the amount of nucleic acids that specifically hybridize to the mutant-type probe.
(4) The method for detecting a BCR-ABL inhibitor resistance-related mutation according to (2), wherein the abundance ratio of wild-type is defined as a proportion of the amount of nucleic acids that specifically hybridize to the wild-type probe and the abundance ratio of mutant-type is defined as a proportion of the amount of nucleic acids that specifically hybridize to the mutant-type probe with respect to the amount of nucleic acids determined using a probe that specifically hybridize to the nucleic acids amplified from the region excluding the mutation site.
(5) The method for detecting a BCR-ABL inhibitor resistance-related mutation according to (3) or (4), wherein a nucleic acid amplification reaction is conducted using a primer having a fluorescence label in the amplifying step, and the amount of nucleic acids that specifically hybridize to the wild-type probe, the amount of nucleic acids that specifically hybridize to the mutant-type probe, and the amount of the amplified nucleic acids are expressed as values based on absolute varies of fluorescence intensity.
(6) The method for detecting a BCR-ABL inhibitor resistance-related mutation according to (1), wherein the mutation site corresponds to a BCR-ABL kinase domain in a protein encoded by the BCR-ABL fusion gene.
(7) The method for detecting a BCR-ABL inhibitor resistance-related mutation according to (1), wherein the mutation site corresponds to one of or both of a site of substitution mutation at position 315 of the BCR-ABL kinase domain from threonine (wild type) to isoleucine (mutant type) and a site of substitution mutation at position 253 of the BCR-ABL kinase domain from tyrosine (wild type) to histidine (mutant type).
(8) The method for detecting a BCR-ABL inhibitor resistance-related mutation according to (7), wherein

a wild-type probe comprising CTATATCATCACTGAGTTCATG (SEQ ID NO: 1) corresponding to the wild-type and a mutant-type probe comprising CTATATCATCATTGAGTTCATG (SEQ ID NO: 2) corresponding to mutant-type nucleic acids are used for the site of substitution mutation at position 315 of the BCR-ABL kinase domain, and

a wild-type probe comprising GCCAGTACGGGGAGGTGTAC (SEQ ID NO: 3) corresponding to wild-type nucleic acids and a mutant-type probe comprising GCCAGCACGGGGAGGTGTAC (SEQ. ID NO: 4) corresponding to mutant-type nucleic acids are used for the site of substitution mutation at position 253 of the BCR-ABL kinase domain.

(9) A data acquisition method for predicting BCR-ABL inhibitor resistance, comprising the step of determining BCR-ABL inhibitor resistance of a subject based on genotype determined for the BCR-ABL fusion gene contained in a biological sample collected from the subject in accordance with the method for detecting a BCR-ABL inhibitor resistance-related mutation according to any one of (1) to (8).

This description includes part or all of the content as disclosed in the description and/or drawings of Japanese Patent Application No. 2014-212228, which is a priority document of the present application.

### Effects of Invention

In the method for detecting a BCR-ABL inhibitor resistance-related mutation of the present invention, a region including a fusion site and a mutation site related to BCR-ABL inhibitor resistance in the BCR-ABL fusion gene is amplified so as to determine genotypes of amplified nucleic acids. Therefore, the genotype of the mutation site can be determined with high accuracy according to the method for detecting a BCR-ABL inhibitor resistance-related mutation of the present invention.

In addition, according to the data acquisition method for predicting BCR-ABL inhibitor resistance of the present invention, information on the genotype determined with high accuracy for the mutation site related to the BCR-ABL inhibitor resistance is utilized, thereby making it possible to predict BCR-ABL inhibitor resistance with high accuracy.

### DESCRIPTION OF EMBODIMENTS

According to the method for detecting a BCR-ABL inhibitor resistance-related mutation of the present invention and the data acquisition method for predicting BCR-ABL inhibitor resistance using the same (hereafter collectively referred to as the method of the present invention), at first, a region including a fusion site and a region including a mutation site (e.g., a mutation site in the BCR-ABL kinase domain) related to BCR-ABL inhibitor resistance in the BCR-ABL fusion gene is amplified using a biological sample collected from a subject. Then, the genotype of the mutation site related to the BCR-ABL inhibitor resistance is determined using amplified nucleic acids so as to determine BCR-ABL inhibitor resistance of the subject based on the determined genotype.

Here, the subject may be a healthy individual or a patient affected with an arbitrary disease that can be treated with a BCR-ABL inhibitor. An arbitrary disease that can be treated with a BCR-ABL inhibitor is not particularly limited. Examples of such disease include chronic myelogenous leukemia (CML), Philadelphia chromosome-positive acute lymphoblastic leukemia (Ph⁺ ALL), KIT (CD117)-positive gastrointestinal stromal tumor (GIST) for imatinib that is a BCR-ABL inhibitor. The subject is particularly preferably a patient affected with chronic myelogenous leukemia. In addition, the subject may be a patient who has started to receive a BCR-ABL inhibitor or who is scheduled to start administration of a BCR-ABL inhibitor. The BCR-ABL inhibitor used herein is not limited to imatinib. Examples thereof include nilotinib and dasatinib.

In particular, a subject having the BCR-ABL fusion gene including a partial translocation between chromosome 9 and chromosome 22 is evaluated for BCR-ABL inhibitor resistance due to a mutation of the BCR-ABL kinase domain of the BCR-ABL fusion gene according to the method of the present invention. Therefore, the subject is designated as having the BCR-ABL fusion gene including a translocation between chromosome 9 and chromosome 22.

In addition, examples of a subject-derived biological sample include, but are not particularly limited to, blood, plasma, serum, spinal fluid, pancreatic fluid, urea, feces, and tissue fluid, Blood, plasma, and serum are particularly preferably used as biological samples.

The expression “amplifying a region including a fusion site and a mutation site related to BCR-ABL inhibitor resistance in the BCR-ABL fusion gene using a biological sample” means extracting total RNA or mRNA from cells contained in a biological sample and conducting a nucleic acid amplification reaction, using as a template, cDNA obtained via reverse transcription of extracted total RNA or mRNA. Examples of nucleic acid extraction means include, but are not particularly limited to, RNA extraction methods using phenol/chloroform, ethanol, sodium hydroxide, CTAB, and the like.

A region including a fusion site and a mutation site related to BCR-ABL inhibitor resistance contained in a BCR-ABL fusion gene is amplified in the nucleic acid amplification reaction. In addition, other regions may also be amplified during the reaction. Examples of a nucleic acid amplification reaction that can be appropriately used include, but are not particularly limited to, polymerase chain reaction (PCR), loop-mediated isothermal amplification (LAMP), transcription-mediated amplification (TMA), transcription-reverse transcription concerted reaction (TRC), strand displacement amplification (SDA), and isothermal and chimeric primer-initiated amplification of nucleic acids (ICAN). For example, in the case of PCR, a pair of primers that flank a region to be amplified are designed and a reagent containing a polymerase, a substrate, and the like is prepared. Then, a nucleic acid amplification reaction is conducted using, as a template, the cDNA obtained via reverse transcription of total RNA or mRNA extracted from a biological sample described above under predetermined temperature cycle conditions so that a region of interest can be specifically amplified using the pair of primers. Note that the nucleic acid amplification reaction is conducted in a reaction system including a buffer necessary for nucleic acid amplification/labeling, thermostable DNA polymerase, primers specific to a region to be amplified, labeled nucleotide triphosphate (specifically nucleotide triphosphate labeled with fluorescence or the like), nucleotide triphosphate, magnesium chloride, and the like.

In particular, it is desirable to add a label in order to identify an amplified region in a nucleic acid amplification reaction. A method for labeling an amplified nucleic acid is not particularly limited. For example, a method in which primers used for amplification reaction are preliminarily labeled or a method in which a labeled nucleotide is used as a substrate in an amplification reaction may be employed. Examples of a labeling substance that can be used include, but are not particularly limited to, radioisotopes, fluorochromes, and organic compounds such as digoxigenin (DIG) and biotin.

Specifically, when PCR is used, a pair of primers that are designed to flank a “region including a fusion site and a mutation site related to BCR-ABL inhibitor resistance contained in a BCR-ABL fusion gene” is used so that the region can be specifically amplified in accordance with an ordinary method. Alternatively, it is also possible to specifically amplify the region using a different nucleic acid amplification method other than PCR in accordance with the principle of a nucleic acid amplification reaction to b employed.

More specifically, a pair of primers used for PCR may be designed based on a region from the BCR gene in the BCR-ABL fusion gene and a region from the ABL gene in the BCR-ABL fusion gene, respectively. Here, such pair of primers includes so-called forward and reverse primers (each hybridizing to a different strand of double-strand DNA). A forward primer may be designed based on a region from the BCR gene or a region from the ABL gene in the BCR-ABL fusion gene, and a reverse primer may be designed based on the other one.

In addition, the mutation site related to BCR-ABL inhibitor resistance is a site for mutation involving an amino acid mutation in, for example, the BCR-ABL kinase domain, which correlates with a phenomenon in which BCR-ABL inhibitor treatment effects are reduced. The phenomenon in which BCR-ABL inhibitor treatment effects are reduced means that a BCR-ABL inhibitor shows reduced effects of inhibiting tyrosine kinase activity of the BCR-ABL fusion protein. A mutation related to BCR-ABL inhibitor resistance is an amino acid substitution mutation in the BCR-ABL kinase domain or the like. Specific examples of mutations of the BCR-ABL kinase domain related to BCR-ABL inhibitor resistance include M237V, I242T, M244V, K247R, L248V, G250E/R, Q252R/H, Y253F/H, E255K/V, E258D, W261L, L273M, E275K/Q, D276G, T277A, E279K, V280A, V289A/I, E292V/Q, I293V, L298V, V299L, F311L/I, T315I, F317L/V/I/C, Y320C, L324Q, Y342H, M343T, A344V, A350V, M351T, E355D/G/A, F359V/I/C/L, D363Y, L364I, A365V, A366G, L370P, V371A, E373K, V379I, A380T, F382L, L384M, L387M/F/V, M388L, Y393C, H396P/R/A, A397P, S417F/Y, I418S/V, A433T, S438C, E450K/G/A/V, E453G/K/V/Q, E459K/V/G/Q, M472I, P480L, F486S, and E507G. Note that in the above mutations, each numerical value corresponds to a mutation site, provided that the numerical value is counted from 1 corresponding to N-terminal methionine of a BCR-ABL fusion protein.

T315I and Y253H are particularly preferably used as mutation sites related to BCR-ABL inhibitor resistance in the BCR-ABL kinase domain. In other words, it is preferable to design a pair of primers in a manner such that the primers flank a region including a fusion site and T315I and/or Y253H related to imatinib resistance contained in the BCR-ABL fusion gene.

The Philadelphia chromosome is a small chromosome derived from a translocation t(9; 22)(q34; q11), in which the c-abl gene located at 9q34 is fused to the her (breakpoint cluster region) gene located at 22q11. The bcr gene has cleavage sites which are concentrated on intron 1 (minor bcr, m-bcr) or intron 2 or 3 (major bcr, M-bcr) and it is fused to a part of c-abl via head-to-tail fusion. In a case in which M-bcr has a cleavage point, b2-a2 mRNA or b3-a2 mRNA in which her exon 2(h2) or exon 3(b3) is ligated to a2 is transcribed, resulting in production of a p210BCR-ABL fusion protein. In a case in which m-bcr has a cleavage point, B1-a2 mRNA in which exon 1 (B1) is ligated to exon 2 (a2) of c-abl, resulting in production of a p190BCR-ABL fusion protein.

A partial sequence of b2-a2 mRNA is shown as a DNA sequence in SEQ ID NO: 5, a partial sequence of b3-a2 mRNA is shown as a DNA sequence in SEQ ID NO: 6, and a partial sequence of B1-a2mRNA is shown as a DNA sequence in SEQ ID NO: 7. In addition, a DNA sequence including a region encoding a BCR-ABL fusion protein, which is contained in b2-a2 mRNA, is shown in SEQ ID NO: 8.

Based on the above findings, it is possible to design appropriate primers for amplifying a region including a fusion site and a mutation site related to BCR-ABL inhibitor resistance contained in a BCR-ABL fusion gene. Specifically, primers can be designed in the following manner.

At first, a mutation to be detected is decided (e.g., T315I). Next, regarding position designing, a forward primer is designed based on a region corresponding to the BCR-ABL fusion gene in the Bcr gene sequence (SEQ ID NO: 9 or 10). A region ranging from the 5′ terminus to position 3304 in SEQ ID NO: 9 or 10 is a region corresponding to a BCR-ABL fusion gene, in addition, a reverse primer is designed based on a region corresponding to a BCR-ABL fusion gene in the Abl gene sequence (SEQ ID NO: 11) in a manner such that the reverse primer serves as a complementary strand on the 3′ terminal side of a mutation site (e.g., T315I). A region ranging from position 445 to the 3′ terminus in SEQ ID NO: 11 corresponds to the BCR-ABL fusion gene. It is also possible to design a forward primer and a reverse primer based on the Abl gene sequence and the Bcr gene sequence, respectively.

As a method for designing primers, an ordinary design method can be used based on nucleotide length, Tm and GC contents, and the like as indexes.

The length of a region to be amplified with a pair of primers is not particularly limited as long as the region includes a fusion site and a mutation site. It is, for example, 10 to 10 k nucleotides, preferably 50 to 6 k nucleotides, more preferably 500 to 3 k nucleotides, and most preferably 800 to 2 k nucleotides. When the length of the region to be amplified falls within the above range, the region can include a fusion site and a mutation site related to BCR-ABL inhibitor resistance contained in the BCR-ABL fusion gene.

A “region including a fusion site and a mutation site related to BCR-ABL inhibitor resistance contained in a BCR-ABL fusion gene” can be specifically amplified using a pair of primers designed as described above. In other words, when a pair of primers designed as described above are used, a nucleic acid amplification reaction does not proceed in the absence of translocation between chromosomes 9 and 22. In such case, amplified fragments would not be confirmed.

Next, the genotype of a mutation site related to the BCR-ABL inhibitor resistance is determined using amplified nucleic acids in the data acquisition method according to the present invention. A method of genotyping is not particularly limited. Genotyping may be carried out based on an arbitrary method or theory. An example of a method of genotyping is a method using probes that specifically hybridize to a wild-type allele and a mutant-type allele at a mutation site. A method of genotyping is not limited to a method using probes. Examples thereof include a method for determining the nucleotide sequence of an amplified fragment and a method such as PCR employing a nucleic acid amplification reaction.

It is particularly preferable to determine the genotype of a mutation site related to the BCR-ABL inhibitor resistance contained in amplified nucleic acids using a DNA microarray (sometimes referred to as a DNA chip) having a base material, on which probes are immobilized. This is because accurate genotyping can be conducted using a DNA microarray, on which probes are immobilized, by simple and convenient procedures.

More specifically, examples of probes for determining the genotype of T315I as a mutation site include a wild-type probe (CTATATCATCACTGAGTTCATG: SEQ ID NO: 1) corresponding to wild-type and a mutant-type probe (CTATATCATCATTGAGTTCATG: SEQ ID NO: 2) corresponding to mutant-type. In addition, examples of probes for determining the genotype of Y253H as a mutation site include a wild-type probe (GCCAGTACGGGGAGGTGTAC: SEQ ID NO: 3) corresponding to wild-type and a mutant-type probe (GCCAGCACGGGGAGGTGTAC: SEQ ID NO: 4) corresponding to mutant-type. The lengths of a wild-type probe and a mutant-type probe are determined to be 22 mer and 20 mer, respectively; however, the nucleotide lengths are not limited. The probe length can be, for example, 10 mer to 50 mer. It is preferably 15 mer to 40 mer, more preferably 20 mer to 30 mer, and most preferably 20 mer to 25 mer. As described above, genotyping probes having any lengths may be designed appropriately based on the nucleotide sequence of the Abl gene.

Genotyping probes are preferably nucleic acids and more preferably DNAs. DNAs include both double-strand and single-strand DNAs. Preferably single-strand DNAs are used. Genotyping probes can be obtained via chemical synthesis using a nucleic acid synthesizer. Examples of a nucleic acid synthesizer that can be used include apparatuses called a DNA synthesizer, a fully automated nucleic acid synthesizer, an automated nucleic acid synthesizer, and the like.

It is preferable to use genotyping probes in the form of a microarray (e.g., DNA chip) in which the 5′ terminus of each probe is immobilized a support. Any material known in the art can be used for the support without particular limitation. In addition, it is preferable to use a support having a carbon layer and a chemical modification group on the surface thereof.

A support having a fine planar structure is particularly preferably used. The shape of a support may be determined as, for example, a rectangular, square, or circle shape without limitation. In general, a square support having a side length of 1 to 75 mm, preferably 1 to 10 mm, and more preferably 3 to 5 mm is used. For the ease of production of a support having a fine planar structure, it is preferable to use a base material comprising a silicon material or a resin material. It is particularly preferable to use a support that is formed with a base material comprising single-crystal silicon and has a carbon layer and a chemical modification group on the surface thereof.

It is possible to immobilize probes to a support by a method in which a spotting solution is prepared by dissolving probes in a spotting buffer, the spotting solution is dispensed on a 96-well or 384-well plastic plate, and the dispensed solution is spotted on a support using a spotting device or the like, can be employed. Such method is not particularly limited, it is also possible to manually spot a spotting solution using a micropipetter.

After spotting, it is preferable to conduct incubation so as to allow a reaction, during which probes bind to a support, to proceed. Incubation is conducted at usually −20° C. to 100° C. and preferably 0° C. to 90° C. for usually 0.5 to 16 hours and preferably 1 to 2 hours, it is desirable to conduct incubation in a highly humid atmosphere at a humidity of, for example, 50% to 90%, it is preferable to conduct washing after incubation in order to remove DNAs that are not bound to the support using a washing liquid (e.g., 50 mM TBS/0.05% Tween 20, a 2×SSC/0.2% SDS solution, or ultrapure water).

The genotype of a mutation site related to BCR-ABL inhibitor resistance in a subject can be determined using a DNA microarray prepared in the above manner. Specifically, a reaction of hybridization between amplified nucleic acids and probes is conducted as described above so that the amount of nucleic acids hybridizing to probes can be determined by detecting, for example, a label. In a case in which, for example, a fluorescence label is used, the intensity of a labeled-derived signal can be quantified by detecting fluorescence signals using a fluorescence scanner and analyzing the signals using image analysis software. In addition, amplified nucleic acids hybridizing to probes may be quantitatively determined by creating a standard curve using, for example, a sample comprising a known amount of DNA. Preferably, a hybridization reaction is conducted under stringent conditions. The term “stringent conditions” refers to conditions under which a specific hybrid is formed while a non-specific hybrid is not formed. Such conditions include conducting a hybridization reaction at, for example, 50° C. for 16 hours, followed by washing with 2×SSC/0.2% SDS at 25° C. for 10 minutes and with 2×SSC at 25° C. for 5 minutes. Alternatively, the hybridization temperature can be set to 42° C. to 54° C. when the salt concentration is 0.5×SSC. When the probe chain length is short, it is more preferable to set the hybridization temperature to 42° C. to 48° C. When the probe chain length is long, it is further preferable to set the hybridization temperature to 46° C. to 54° C. Note that as the salt concentration increases, the specific hybridization temperature increases, while on the other hand, as the salt concentration decreases, the specific hybridization temperature decreases.

Here, in addition to probes for determining the genotype of a mutation site, a probe that hybridizes to a region other than the region including the mutation site related to BCR-ABL inhibitor resistance in amplified nucleic acids may be immobilized to a DNA microarray. Such probe can hybridize to amplified nucleic acids regardless of the genotype of the mutation site. Accordingly, it is possible to determine whether or not nucleic acids of interest can be amplified in a nucleic acid amplification reaction using a DNA microarray to which the probe is immobilized.

According to the method of the present invention, based on the amount of the amplified nucleic acids hybridizing to probes designed for a mutation site related to BCR-ABL inhibitor resistance, the genotype of the mutation site is determined. In particular, since a region including a fusion site and a mutation site related to BCR-ABL inhibitor resistance contained in a BCR-ABL fusion gene is amplified in the method of the present invention, it is possible to determine the genotype of a mutation site of the BCR-ABL fusion gene without the influence of the genotype of the corresponding mutation site of the ABL gene on chromosome 9.

In particular, it is possible to calculate a ratio of the expression level of the wild-type BCR-ABL fusion gene and the expression level of the mutant-type BCR-ABL fusion gene by determining the genotype of a mutation site for nucleic acids that have been amplified using, as a template, cDNA obtained via reverse transcription of mRNA using a biological sample collected from a subject. In other words, in such case, it is possible to estimate abundance of a cell (clone) population resistant to a BCR-ABL inhibitor with respect to abundance of cells sensitive to a BCR-ABL inhibitor for a biological sample collected from a subject. Accordingly, it is possible to appropriately determine a treatment policy for an arbitrary disease that can be treated with a BCR-ABL inhibitor (e.g., CML, Ph⁺ ALL, or GIST) regarding whether to continue administration of a BCR-ABL inhibitor or to switch to a different therapeutic agent.

More specifically, abundance ratio of wild-type and/or mutation-type among the amplified nucleic acids is calculated using a wild-type probe for wild-type and a mutant-type probe for mutant-type regarding the above mutation site. When the abundance ratio of the wild-type is at or below a certain level or the abundance ratio of the mutant-type is at or above a certain level, it is possible to determine that, for example, a risk of the recurrence of a disease is high. Here, it is possible to appropriately predetermine, as a threshold for the mutant-type abundance, a value indicating a high risk of the recurrence of a disease, a value indicating no effects of BCR-ABL inhibitor administration, a value indicating a timing for switching from a BCR-ABL inhibitor to a different agent, or the like.

In one example, the abundance ratio of wild-type or mutant-type can be determined in the following manner. Specifically, abundance ratio of wild-type is defined as a proportion of the amount of nucleic acids that specifically hybridize to the wild-type probe, and abundance ratio of mutant-type is defined as a proportion of the amount of nucleic acids that specifically hybridize to the mutant-type probe, with respect to the sum of the amount of nucleic acids that specifically hybridize to the wild-type probe and the amount of nucleic acids that specifically hybridize to the mutant-type probe. Alternatively, abundance ratio of wild-type may be defined as a proportion of the amount of nucleic acids that specifically hybridize to the wild-type probe, and abundance ratio of mutant-type may be defined as a proportion of the amount of nucleic acids that specifically hybridize to the mutant-type probe, with respect to the amount of nucleic acids determined using a probe that specifically hybridize to a region other than the mutation site.

In any case, abundance of wild-type nucleic acids is defined as the amount of the wild-type BCR-ABL fusion gene, and abundance of mutant-type nucleic acids is defined as the amount of the mutant-type BCR-ABL fusion gene, with respect to the sum of the amount of amplified nucleic acids, that is to say, the sum of the wild-type BCR-ABL fusion gene and the mutant-type BCR-ABL fusion gene. Accordingly, it is possible to rationally estimate abundance of BCR-ABL inhibitor resistance clones contained in a biological sample collected from a subject.

### EXAMPLES

Hereafter, the present invention is described in detail with reference to the Examples below. However, the scope of the present invention is not limited to the Examples.

### Example 1

### <Preparation of Specimens>

Five types of specimens summarized in the table below were prepared in this Example.

TABLE 1

| | | | Mutation type | | Mutation type | |
|---|---|---|---|---|---|---|
| | | | T315I | | Y253H | |
| | | BCR-ABL | Wild | Mutation | Wild | Mutation |
| | | Fusion | type | type | type | type |
| Specimen 1 | K562 | + | + | − | + | − |
| Specimen 2 | HL60 | − | + | − | + | − |
| Specimen 3 | BCRABL/wild type | + | + | − | + | − |
| Specimen 4 | BCRABL/T315I mutation | + | − | + | + | − |
| Specimen 5 | BCRABL/Y253H mutation | + | + | − | − | + |

### <Method for Preparing Specimen 1>

The K562 cell line was cultured and the cell concentration was confirmed to be 1.25×10⁶ cells/mL using TC20 automated cell counter (Bio-Rad Laboratories, Inc.). The culture solution (10 mL) was sampled in a 15-mL Falcon tube and centrifuged at 300×g for 5 minutes. Thus, a pellet was obtained. The supernatant was completely removed in a careful manner, followed by extraction of RNA using an RNeasy Mini Kit (QIAGEN). The RNA concentration was determined using NanoDrop 2000 (Thermo Fisher Scientific Inc.).

### <Method for Preparing Specimen 2>

As in the case of preparation of specimen 1 the HL60 cell line was cultured, followed by extraction of RNA using an RNeasy Mini Kit (QIAGEN). The RNA concentration was determined using NanoDrop 2000 (Thermo Fisher Scientific Inc.).

### <Method for Preparing Specimen 3>

RT-PCR was performed using the following primers for 2 mg of total RNA extracted from the K562 cell [Primer sequence 5 (CCGGAATTCTCCGCTGACCATCAATAAGGA: SEQ ID NO: 12); Primer sequence 6 (ATAAGAATGCGGCCGCACGTCGGACTTGATGGAGAACT: SEQ ID NO: 13)]. A 1203-bp cDNA fragment was amplified using a 5′ BCR-specific primer and a 3′ ABL-specific primer.

The PCR product was ligated to a pcDNA3.1 (+) cloning vector (Invitrogen) and transformed into E. coli DH5α competent cells (Toyobo Co., Ltd.), The inserted PCR product was sequenced. Thus, it was confirmed that the wild-type sequence had been inserted therein.

### <Method for Preparing Specimen 4>

A point mutation at position 315 from threonine to isoleucine was introduced into the wild-type clone vector obtained for the specimen 3 using Site-Directed Mutagenesis (Toyobo Co., Ltd.). The following primer sequences were used for PCR: primer sequence 7 (CCCCGTTCTATATCATCATTGAGTTCATGACCTACG: SEQ ID NO: 14) and primer sequence 8 (CGTAGGTCATGAACTCAATGATGATATAGAACGGGG: SEQ ID NO: 15). Table 2 lists the reaction solution composition (unit: μL).

TABLE 2

| | |
|---|---|
| 10 × Buffer for KOD-Plus- Ver. 2 | 5 |
| 2 mN dNTPs | 5 |
| 25 mM MgSO₄ | 3 |
| FW Primer (10 μM) | 1.5 |
| RV Primer (10 μM) | 1.5 |
| KOD-Plus- (1 U/μl) | 1 |
| Template wild-type clone vector | 6 |
| RNase Free water | 27 |
| Total volume | 50 |

PCR was performed under the following conditions. In other words, 10 cycles of 95° C. for 2 minutes, 98° C. for 2 minutes, and 68° C. for 10 minutes were conducted.

The wild-type clone vector used as a template was fragmented using restriction enzyme DpnI (Toyobo Co., Ltd.) and the PCR product incorporating the mutation of interest was transformed into E. coli DH5α competent cells (Toyobo Co., Ltd.). The inserted PCR product was sequenced. Thus, it was confirmed that the T315I mutant-type sequence had been inserted therein.

### <Method for Preparing Specimen 5>

A point mutation at position 253 from tyrosine histidine was introduced into the wild-type clone vector obtained for the specimen 3 using Site-Directed Mutagenesis (Toyobo Co., Ltd.). The following primer sequences were used for PCR: primer sequence 9 (CAAGCTGGGCGGGGGCCAGCACGGGGAGGTGTACGAGG: SEQ ID NO: 16); and primer sequence 10 (CCTCGTACACCTCCCCGTGCTGGCCCCCCCCAGCTTG: SEQ ID NO: 17). PCR was performed under the conditions for preparing the specimen 4 and the inserted PCR product was sequenced. Thus, it was confirmed that the mutant-type Y253H sequence had been inserted therein.

### <Preparation of Chips>

A probe (probe 1) for detecting a fusion site of the BCR-ABL fusion gene and probes (probes 2 and 3) for detecting T315I as a BCR-ABL fusion gene mutation were prepared. The following are the nucleotide sequences of the probes.

(SEQ ID NO: 18)

| | |
|---|---|
| Probe 1 (fusion): | CCCTTCAGCGGCCAGTAGCATCTGA |

(SEQ ID NO: 1)

| | |
|---|---|
| Probe 2 (wild type): | CTATATCATCACTGAGTTCATG |

(SEQ ID NO: 2)

| | |
|---|---|
| Probe 3 (mutant type): | CTATATCATCATTGAGTTCATG |

### [Example 1-1] Evaluation of Primer Sets

In this Example, a primer set A (primers 1 and 2) for specifically amplifying a region including a fusion site and a mutation site of the BCR-ABL fusion gene was used. The following are the nucleotide sequences of primers 1 and 2. Note that Cy5 was added to the primer 2.

(SEQ ID NO: 19; Y = C or T)
Primer 1: TCCGCTGACCATCAAYAAGGA
(SEQ ID NO: 20)
Primer 2: ACGTCGGACTTGATGGAGAACT

RT-PCR was performed using the primer set A in a reaction solution having the composition listed in Table 3 below (unit: μL) so that a region including a fusion site and a mutation site of the BCR-ABL fusion gene was amplified. In this Example, human RNA extracted from the specimen 1 was used as a template.

TABLE 3

| | |
|---|---|
| 2 X One Step SYBR RT-PCR Buffer 4 | 25 |
| RNase Free water | 16 |
| Primer 1 (10 μM) | 2 |
| Primer 2 (10 μM) | 4 |
| TaKaRa EX Taq HS Mix | 3 |
| PrimeScript 1 step Enzyme Mix | 1 |
| Template RNA | 1 |
| Total volume | 50 |

RT-PCR was performed by conducting a reverse transcription reaction at 42° C. for 15 minutes and 95° C. for 10 seconds and then an amplification reaction for 33 cycles of 95° C. for 30 seconds, 60° C. for 30 seconds, and 72° C. for 3 minutes, followed by 72° C. for 5 minutes.

After the termination of the reaction, the reaction solution and the hybridization buffer (2×SSC/0.2% SDS/0.2 nM Cy5-labeled oligo DNA (Sigma-Aldrich Co. LLC.)) were mixed at 1:1, 3 μL of the mixture was sampled and added dropwise onto a protrusion formed at the center of a hybridization cover. The cover was placed over a chip. A reaction was conducted in an hybridization chamber (Toyo Kohan Co.; Ltd.) at 48° C. for 1 hour.

After the hybridization reaction, a stainless-steel holder for washing was immersed in a 1×SSC/0.1% SDS solution, the hybridization cover was removed from the chip, and the chip was set to the holder. The holder was shaken several times in the vertical direction and then immersed in the 1×SSC solution (room temperature) until fluorescence intensity of the chip was detected.

A cover glass was placed over the chip. Then, fluorescence intensity was detected using FLA8000 (Fujifilm Corporation) at a pixel size of 5 μm and PMT 80%. Data obtained by FLA8000 were analyzed using the software (ArrayGauge Ver. 2.1; Fujifilm Corporation).

For comparison, RT-PCR, the hybridization reaction, and fluorescence detection were conducted in the above manner except that a primer set B (primers 3 and 4) for amplifying a BCR-ABL fusion gene mutation was used instead of the primer set A. The following are the nucleotide sequences of primers 3 and 4.

(SEQ ID NO: 21)
Primer 4: AAGACCTTGAAGGAGGACACCAT
(SEQ ID NO: 22)
Primer 4: ACGTCGGACTTGATGGAGAACT

Table 4 shows the results of determination of fluorescence intensity in the case of using the primer set A and the case of using the primer set B. As shown in Table 4, signals were obtained for both the fusion site and the mutation site for the specimen 1 having the BCR-ABL fusion gene in the case of using the primer set A. Meanwhile, in the case of using the primer set B, the mutation site was detectable; however, the fusion site was not detectable from the specimen 1 having the BCR ABL fusion gene. Accordingly, it was revealed that both a fusion site and a mutation site can be detected using the primer set A. Note that the undetectable value was set to not more than 43. The value was calculated from the results of analysis of fluorescence signals in the background region containing no signals.

TABLE 4

| | | | Mutation site | |
|---|---|---|---|---|
| | | Fusion | Wild | Mutation |
| | Template specimen | site | type | type |
| Primer set A | Specimen 1: 10 ng | 142.5 | 1781.2 | 155.6 |
| Primer set B | | 39.1 | 156.5 | 43.8 |

### [Example 1-2] Evaluation of Primer Sets

In this Example, a DNA chip, on which a probe for detecting a Y253H mutation as well as a probe for detecting a T315I mutation had been fixed for mutation sites of the BCR-ABL fusion gene, was used for detecting a mutation site in the BCR-ABL fusion gene contained in a specimen. In this Example, human RNA (100 ng) extracted from the cell line of the specimen 2 and human DNA (1 ng) extracted from the cell line of the specimen 5 were used as templates.

In this Example, probes (probes 4 and 5) for detecting Y253H as a BCR-ABL fusion gene mutation was designed, in addition to probes 1 to 3 so that a DNA chip, to which the probes 1 to 5 were fixed, were prepared.

(SEQ ID NO: 3)
Probe 4 (wild type: Y253HGCCAGTACGGGGAGGTGTAC)
(SEQ ID NO: 4)
Probe 5 (mutant type: GCCAGCACGGGGGAGGTGTAC)

Table 5 shows the results of determination of fluorescence intensity in the case of using the primer set A as described in Example 1-1. As shown in Table 5, in the case of the use of the primer set A, it is possible to collectively examine a fusion site and a plurality of mutation sites (at least Y253H and T315I).

TABLE 5

| | | | T315I mutation site | | Y253H mutation site | |
|---|---|---|---|---|---|---|
| | Template specimen | Fusion site | Wild type | Mutation type | Wild type | Mutation type |
| Primer set A | Specimen 2: 100 ng | 129.9 | 1710.3 | 121.7 | 866.1 | 1693.3 |
| | Specimen 5: 1 ng | | | | | |

### [Example 2-1] Evaluation of Primer Sets

In this Example, it was verified whether it would be possible to distinguish the specimen 1 having the BCR-ABL fusion gene and the specimen 2 having no BCR-ABL fusion gene using the primer sets A and B. When the genotype of a mutation site in the BCR-ABL fusion gene in a biological sample from a subject is determined in practice, as the genotype of the corresponding site in the normal cell-derived c-ABL gene is detected simultaneously, the rate of mutation in the BCR-ABL fusion gene might be estimated at a level lower than the actual level. In this Example, it was verified whether the use of the primer sets A and B would result in such inconvenience.

In this Example, fluorescence intensity was determined from a DNA chip using the specimens 1 and 2 as described in Example 1-1. Table 6 shows the results. As shown in Table 6, in the case of using the primer set A, fluorescence signals were detected for the specimen 1 having the BCR-ABL fusion gene, indicating that it was possible to distinguish the wild type at the mutation site, Meanwhile, in the case of using the primer set A, fluorescence signals were not detected for the specimen 2 having no BCR-ABL fusion gene, indicating that there was no BCR-ABL fusion gene-derived mutation. As stated above, it was revealed that the specimen 1 and the specimen 2 can be clearly distinguished from each other in the case of using the primer set A.

On the other hand, in the case of using the primer set B, fluorescence signals were detected also for the specimen 2 having no BCR-ABL fusion gene. In this case, it was impossible to distinguish the specimen 1 from the specimen 2.

TABLE 6

| | | | Mutation site | |
|---|---|---|---|---|
| | | Fusion | Wild | Mutation |
| | Template specimen | site | type | type |
| Primer set A | Specimen 1: 10 ng | 142.5 | 1781.2 | 155.6 |
| | Specimen 2: 10 ng | 40.2 | 39.5 | 40.9 |
| Primer set B | Specimen 1: 10 ng | 39.1 | 156.5 | 43.8 |
| | Specimen 2: 10 ng | 34.9 | 102.7 | 37.2 |

### [Example 2-2] Evaluation of Primer Sets

In this Example, the total mixed amount of the specimens 3 and 4 was set to 10 ng. Mixtures containing the specimens at ratios of 100:0, 50:50, and 0:10 were used as templates. PCR, the hybridization reaction using a DNA chip, and the subsequent fluorescence detection were conducted as described in Example 1-1.

Table 7 lists the results of fluorescence intensity determination in the case of using the primer set A and in the case of using the primer set B for the specimens 3 and 4 containing no normal cell-derived c-ABL gene. As shown in Table 7 below, in both cases, there was a correlation between the ratio of the specimens mixed as templates and the mutation rate based on signal intensity.

TABLE 7

| | | | Mutation site | |
|---|---|---|---|---|
| | | Fusion | Wild | Mutation |
| | Template specimen | site | type | type |
| Primer set A | Specimen 3 | 187.1 | 8211.4 | 720.7 |
| | Specimen 3/Specimen 4 | 310.4 | 4495.6 | 4186.2 |
| | (50/50) | | | |
| | Specimen 4 | 546.9 | 363.0 | 9110.5 |
| Primer set B | Specimen 3 | 34.0 | 1297.7 | 72.9 |
| | Specimen 3/Specimen 4 | 40.9 | 1163.5 | 744.5 |
| | (50/50) | | | |
| | Specimen 4 | 34.8 | 53.2 | 915.9 |

### [Example 2-3] Evaluation of Primer Sets

In this Example, a mixture of the specimen 4 (1 ng) and the specimen 2 (100 ng) was used as a template. PCR, the hybridization reaction using a DNA chip, and the subsequent fluorescence detection were conducted as described in Example 1-1.

Table 8 lists the results of determination of fluorescence intensity in the case of using the primer set A and in the case of using the primer set B when the specimen 2 having the normal cell-derived c-ABL gene was included. As shown in table 8 below, in the case of using the primer set A, there was a correlation between the rate of mutation of the BCR-ABL fusion gene used as a template (mutant type: 100%) and the detected signal intensity. Meanwhile, in the ease of using the primer set B, it was revealed that the correlation between the rate of mutation of the BCR-ABL fusion gene used as a template and the detected signal intensity was inverted, indicating that mutation was significantly influenced by the specimen 2 having no BCR-ABL, fusion gene. Accordingly, it was revealed that it is possible to correctly determine mutation at a mutation site without influence of the normal cell-derived c-ABL gene in the case of using the primer set A.

TABLE 8

| | | | Mutation site | |
|---|---|---|---|---|
| | | Fusion | Wild | Mutation |
| | Template specimen | site | type | type |
| Primer set A | Specimen 2: 100 ng | 259.2 | 48.2 | 336.0 |
| Primer set B | Specimen 4: 1 ng | 43.0 | 291.6 | 45.3 |

All publications, patents, and patent applications cited herein are incorporated herein by reference in their entirety.

We claim:

1. A method for detecting a BCR-ABL inhibitor resistance-related mutation, comprising the steps of:
amplifying a region including a fusion site and a mutation site related to BCR-ABL inhibitor resistance contained in a BCR-ABL fusion gene using a biological sample collected from a subject; and
genotyping of the mutation site related to BCR-ABL inhibitor resistance using nucleic acids amplified in the above step.

2. The method for detecting a BCR-ABL inhibitor resistance-related mutation according to claim 1, wherein abundance ratio of wild-type and/or mutation-type among the amplified nucleic acids is calculated using a wild-type probe for wild-type and a mutant-type probe for mutant-type for the mutation site in the step of genotyping.

3. The method for detecting a BCR-ABL inhibitor resistance-related mutation according to claim 2, wherein the abundance ratio of wild-type is defined as a proportion of the amount of nucleic acids that specifically hybridize to the wild-type probe and the abundance ratio of mutant-type is defined as a proportion of the amount of nucleic acids that specifically hybridize to the mutant-type probe with respect to the sum of the amount of nucleic acids that specifically hybridize to the wild-type probe and the amount of nucleic acids that specifically hybridize to the mutant-type probe.

4. The method for detecting a BCR-ABL inhibitor resistance-related mutation according to claim 2, wherein the abundance ratio of wild-type is defined as a proportion of the amount of nucleic acids that specifically hybridize to the wild-type probe and the abundance ratio of mutant-type is defined as a proportion of the amount of nucleic acids that specifically hybridize to the mutant-type probe with respect to the amount of nucleic acids determined using a probe that specifically hybridize to the nucleic acids amplified from the region excluding the mutation site.

5. The method for detecting a BCR-ABL inhibitor resistance-related mutation according to claim 3, wherein a nucleic acid amplification reaction is conducted using a primer having a fluorescence label in the amplifying step, and the amount of nucleic acids that specifically hybridize to the wild-type probe, the amount of nucleic acids that specifically hybridize to the mutant-type probe, and the amount of the amplified nucleic acids are expressed as values based on absolute values of fluorescence intensity.

6. The method for detecting a BCR-ABL inhibitor resistance-related mutation according to claim 1, wherein the mutation site corresponds to a BCR-ABL kinase domain in a protein encoded by the BCR-ABL fusion gene.

7. The method for detecting a BCR-ABL inhibitor resistance-related mutation according to claim 1, wherein the mutation site corresponds to one of or both of a site of substitution mutation at position 315 of the BCR-ABL kinase domain from threonine (wild type) to isoleucine (mutant type) and a site of substitution mutation at position 253 of the BCR-ABL kinase domain from tyrosine (wild type) to histidine (mutant type).

8. The method for detecting a BCR-ABL inhibitor resistance-related mutation according to claim 7, wherein
a wild-type probe comprising CTATATCATCACTGAGTTCATG (SEQ ID NO: 1) corresponding to wild-type a mutant-type probe and comprising CTATATCATCATTGAGTTCATG (SEQ ID NO: 2) corresponding to mutant-type are used for the site of substitution mutation at position 315 of the BCR-ABL kinase domain, and
a wild-type probe comprising GCCAGTACGGGGAGGTGTAC (SEQ ID NO: 3) corresponding to wild-type and a mutant-type probe comprising GCCAGCACGGGGAGGTGTAC (SEQ ID NO: 4) corresponding to mutant-type are used for the site of substitution mutation at position 253 of the BCR-ABL kinase domain.

9. A data acquisition method for predicting BCR-ABL inhibitor resistance, comprising the step of determining BCR-ABL inhibitor resistance of a subject based on genotype determined for the BCR-ABL fusion gene contained in a biological sample collected from the subject in accordance with the method for detecting a BCR-ABL inhibitor resistance-related mutation according to claim 1.

10. The method for detecting a BCR-ABL inhibitor resistance-related mutation according to claim 4, wherein a nucleic acid amplification reaction is conducted using a primer having a fluorescence label in the amplifying step, and the amount of nucleic acids that specifically hybridize to the wild-type probe, the amount of nucleic acids that specifically hybridize to the mutant-type probe, and the amount of the amplified nucleic acids are expressed as values based on absolute values of fluorescence intensity.

11. A data acquisition method for predicting BCR-ABL inhibitor resistance, comprising the step of determining BCR-ABL inhibitor resistance of a subject based on genotype determined for the BCR-ABL fusion gene contained in a biological sample collected from the subject in accordance with the method for detecting a BCR-ABL inhibitor resistance-related mutation according to claim 2.

12. A data acquisition method for predicting BCR-ABL inhibitor resistance, comprising the step of determining BCR-ABL inhibitor resistance of a subject based on genotype determined for the BCR-ABL fusion gene contained in a biological sample collected from the subject in accordance with the method for detecting a BCR-ABL inhibitor resistance-related mutation according to claim 3.

13. A data acquisition method for predicting BCR-ABL inhibitor resistance, comprising the step of determining BCR-ABL inhibitor resistance of a subject based on genotype determined for the BCR-ABL fusion gene contained in a biological sample collected from the subject in accordance with the method for detecting a BCR-ABL inhibitor resistance-related mutation according to claim 4.

14. A data acquisition method for predicting BCR-ABL inhibitor resistance, comprising the step of determining BCR-ABL inhibitor resistance of a subject based on genotype determined for the BCR-ABL fusion gene contained in a biological sample collected from the subject in accordance with the method for detecting a BCR-ABL inhibitor resistance-related mutation according to claim 5.

15. A data acquisition method for predicting BCR-ABL inhibitor resistance, comprising the step of determining BCR-ABL inhibitor resistance of a subject based on genotype determined for the BCR-ABL fusion gene contained in a biological sample collected from the subject in accordance with the method for detecting a BCR-ABL inhibitor resistance-related mutation according to claim 6.

16. A data acquisition method for predicting BCR-ABL inhibitor resistance, comprising the step of determining BCR-ABL inhibitor resistance of a subject based on genotype determined for the BCR-ABL fusion gene contained in a biological sample collected from the subject in accordance with the method for detecting a BCR-ABL inhibitor resistance-related mutation according to claim 7.

17. A data acquisition Method for predicting BCR-ABL inhibitor resistance, comprising the step of determining BCR-ABL inhibitor resistance of a subject based on genotype determined for the BCR-ABL fusion gene contained in a biological sample collected from the subject in accordance with the method for detecting a BCR-ABL inhibitor resistance-related mutation according to claim 8.
